(19) United States
(12) Patent Application Publication
(10) Pub. No.: US 2021/0353514 A1
(43) Pub. Date: Nov. 18, 2021

(54) SUNSCREEN FORMULATION

(71) Applicant: BEIERSDORF AG, Hamburg (DE)

(72) Inventors: Jayesh PATEL, Chandler, AZ (US); Sherilee Jane BACKMAN, Rye, NY (US)

(21) Appl. No.: 17/287,338

(22) PCT Filed: Oct. 23, 2019
(86) PCT No.: PCT/US2019/057718

§ 371 Date: Apr. 21, 2021

Related U.S. Application Data
(60) Provisional application No. 62/750,248, filed on Oct. 24, 2018.

(51) Int. Cl. A61K 8/27; A61K 8/06; A61K 8/34; A61K 8/37; A61K 8/893; A61Q 17/04

(52) U.S. Cl. CPC A61K 8/27; A61K 8/064; A61K 8/345; A61K 8/347; A61K 8/375; A61K 2800/594; A61Q 17/04; A61K 2800/30; A61K 2800/612; A61K 2800/413; A61K 8/893

(57) ABSTRACT

The present invention relates generally to sun protection compositions for application to the skin, and in particular sunscreen formulations comprising zinc oxide and other components in a water-in-silicone/oil inverted emulsion system.

### FIELD

The present invention relates generally to sun protection compositions for application to the skin, and more specifically to sunscreen formulations comprising zinc oxide and other components in a hybrid water-in-silicone/oil inverted emulsion system.

### BACKGROUND

It is known that the combination of active organic sunscreen agents can provide broad spectrum coverage. However, use of active organic sunscreen agents may be harmful or irritating when applied to skin. There is accordingly a need for a sunscreen formulation having broad spectrum coverage and which does not depend upon organic chemicals.

In particular, what is desired in the art are sunscreen formulations that can achieve high Sun Protection Factor (SPF) of at least 50, and have both the stability and aesthetic qualities as well as replicability on a commercially-viable scale.

### SUMMARY OF THE INVENTION

The present invention addresses this need by providing compositions suitable for use as sunscreens for application on human skin. In some aspects, provided are sunscreen formulations that can achieve a sun protection factor (SPF) value of 50 or greater using 25% by weight or less of zinc oxide as the sunscreen actives. The SPF is measured according to the procedure prescribed by the U.S. Food and Drug Administration (21 CFR 201.327(i)). In certain aspects, provided are sunscreen formulations made up of zinc oxide (e.g., powders or dispersions) and other components in a water-in-silicone/oil inverted emulsion system, i.e., an emulsion system in which a mixture of one or more silicones and one or more non-silicones is the continuous (oil) phase.

In some embodiments, such formulations use only mineral sunscreen actives, and exclude organic actives, to achieve the desired SPF values. Such formulations were unexpectedly observed to have high SPF and Ultra Violet A (UVA) readings given the type and amount of actives used in the formulation. Such formulations also have the water resistance, stability and aesthetic qualities desirable for commercial sunscreen products.

In some aspects, provided is a sunscreen formulation having a SPF value of at least 50, comprising: no more than 25 wt % zinc oxide based on a total weight of the sunscreen formulation, wherein the zinc oxide preferably is surface treated; one or more film formers; and one or more SPF boosters.

In other aspects, provided is a hybrid water-in-silicone/oil inverted emulsion comprising: no more than 25 wt % zinc oxide based on a total weight of the emulsion, wherein the zinc oxide is preferably surface treated; one or more film formers; and one or more SPF boosters.

In some variations of the foregoing, the sunscreen formulation does not include any organic sunscreen actives.

In some variations of the foregoing, the surface-treated zinc oxide is zinc oxide surface treated with ethoxylated alkylsilane, such as triethoxycaprylylsilane.

In another aspect, provided is a sunscreen formulation, comprising: at least one zinc oxide surface treated with triethoxycaprylylsilane, wherein the zinc oxide preferably has a median diameter of the particle number size distribution D50 (50% of the number below this diameter) above 30 nm (as determined by light scattering methods); bis-octyldodecyl dimer dilinoleate/propanediol copolymer; octyldodecyl/glyceryl hydroxy stearate dilinoleate dimethicone copolymer; and preferably at least one SPF booster. In some embodiments, the zinc oxide may have a median particle size (D50) greater than about 1000 nm, greater than about 1500 nm, greater than about 2000 nm, greater than about 2500 nm, or greater than about 3000 nm. In some variations, such sunscreen formulation is a hybrid water-in-silicone/oil inverted emulsion, and has no more than 25 wt % zinc oxide based on a total weight of the sunscreen formulation, wherein the zinc oxide is preferably surface treated.

In certain embodiments of the foregoing, the compositions described may have a water resistance for up to about 40 minutes, preferably up to about 60 minutes or up to about 80 minutes (determined by the procedure prescribed by the U.S. Food and Drug Administration).

In other embodiments that may be combined with any of the foregoing, the compositions described may have a UVA Protection Factor (UVAPF, determined according to the method prescribed by the U.S. Food and Drug Administration, COLIPA 2011) of at least ⅓ of the SPF value. In some embodiments, the compositions described may have a UVAPF of at least 16.7, at least 17, at least 18, at least 19, or at least 20 at a critical wavelength of above 370 nm.

In other variations of the foregoing, the compositions described may be formulated as a lotion, or a cream, or a spray. Such lotion/cream/spray is suitable for use on human skin.

Provided are also methods for manufacturing compositions as set forth above. In one aspect, provided is a method of preparing the compositions described herein, comprising: mixing one or more film formers and one or more SPF boosters to provide a silicone/oil mixture; adding to the first mixture no more than 25 wt % zinc oxide based on a total weight of the final inverted emulsion, wherein the zinc oxide is preferably surface treated; and combining the silicone/oil mixture with an aqueous mixture to provide a hybrid water-in-silicone/oil inverted emulsion. In certain embodiments, the aqueous mixture is introduced into the silicone/oil mixture to provide the water-in-silicone/oil inverted emulsion. In another aspect, provided is a composition produced according to any of the methods described herein.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

As used herein, the word “a” or “plurality” before a noun represents one or more of the particular noun. For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used.

Provided herein are compositions suitable for use as sunscreen compositions. Such sunscreen compositions can be used on human skin. In some aspects, the compositions provided are all mineral sunscreen compositions comprising zinc oxide, and have unexpectedly been found to achieve a Sun Protection Factor (SPF) of at least 50. In certain variations, titanium dioxide can be used in combination with zinc oxide (or even alone) as the mineral sunscreen active ingredients. In certain variations, the sunscreen composition is free of any organic sunscreen actives.

In some aspects, provided is a composition that is formulated as a water-in-silicone/oil inverted emulsion. Such composition comprises no more than 25 wt % zinc oxide (based on a total weight of the composition), wherein the zinc oxide is preferably surface treated. Such composition preferably also comprises film formers and/or SPF boosters, and other ingredients. The sunscreen formulation and the agents used therein, as well as the method of manufacturing such compositions are described in further detail below.

### Inverted Emulsion

In some embodiments, the compositions described herein are formulated as a water-in-silicone/oil inverted emulsion system. The use of such a vehicle was found to have several advantages. Without being bound to any particular theory, the use of such inverted emulsion was unexpected to often enhance the efficacy of the mineral sunscreen actives, and generally performed better than in conventional water-in-oil systems. Another advantage of using such an inverted emulsion may be the enhancement of tactile and textural properties of the final product. Additionally, the inverted emulsion may afford less irritation and eye sting potential than do conventional emulsions. Finally, the inverted emulsion may confer greater water resistance than do conventional systems. The formulation approach of using this type of emulsion in combination with the film formers and other agents described herein may greatly increase the ability to achieve 80-minute water resistance for the final product.

### Sunscreen Actives

In some embodiments, the actives used in the sunscreen formulations described herein are solely mineral actives. In some variations, the formulation excludes any organic actives. Several commercially useful inorganic UV-protective agents are known, notably titanium dioxide, zinc oxide and iron oxide. Titanium dioxide and zinc oxide are approved for use as sunscreens by regulatory authorities such as the U.S. Food and Drug Administration (FDA). Generally speaking, of the two, zinc oxide provides better blockage of UVA wavelengths, and so is the better material to ensure an at least one-third ratio of UVA absorption compared to SPF value (UVAPF), and acceptable critical wavelength to provide broad spectrum protection.

In some embodiments, the mineral actives used are zinc oxide. In some variations, the zinc oxide may be in powder form. In other variations, the zinc oxide may be in a dispersion. In yet other variations, the zinc oxide may be in a slurry or paste form. The effectiveness of sunscreen ingredients may be influenced by a number of factors. It is appreciated in the art that the type and concentration of actives used in a formulation dictate the expectation of the SPF of such a formulation. A person of ordinary skill in the art would therefore expect an increase of SPF by 1-1.5 times simply based on the concentration of zinc oxide used in a formulation. Therefore, it is surprising to achieve a high SPF rating of at least 50 using not more than 25 wt % zinc oxide (but usually at least 2 wt %, e.g., at least 5 wt. %, at least 10 wt. %, at least 15 wt. %, or at least 20 wt. %) in sunscreen formulations as disclosed in the present application.

In certain embodiments, the zinc oxide is surface-treated. Generally speaking, surface treatment usually enhances the quality of zinc oxide dispersions and increases the proportion of zinc oxide that can be successfully loaded into emulsions. If desired, the surface treatment can be selected according to the nature of the intended end product, several options for which are known to those skilled in the art. Exemplary surface treatment agents include, but are not limited to, methicone, hydrogen dimethicone, dimethicone, triethoxycaprylylsilane, jojoba esters, polymethylsilsesquioxane, magnesium myristate (and other metal soaps), stearic acid, stearoyl glutamic acid (and other amino acid treatments), lecithin, silica, sodium polyacrylate, PEG-10 dimethicone, sodium lauroyl aspartate, stearyl triethoxysilane, disodium carboxyethyl siliconate, trimethylsiloxysilicate, dimethicone PEG-3 laurate, stearyl triethoxysilane, disodium carboxyethyl siliconate, trimethylsiloxysilicate, polycaprylylsilsesquioxane, polydiethylsiloxane, triethoxysilylpropyl acetyl hydroxyprolinate, perfluorooctylethyltriethoxysilane, methoxy PEG-10 propyltrimethoxysilane, silanetriol, and any combination thereof.

In some variations, the zinc oxide may be surface treated with ethoxylated alkyl silane. In a certain variation, the zinc oxide may be surface treated with triethoxycaprylylsilane. Examples of surface-treated zinc oxide suitable for use in the formulations described herein include two (or more) types of zinc oxide (and) triethoxycaprylylsilane.

In certain embodiments, the formulations described herein may include one type of surface-treated zinc oxide. In other embodiments, the formulations may include a combination of two or more types of surface-treated zinc oxide. In one embodiment, a combination of surface-treated zinc oxides was used in the formulation, and was chosen to balance efficacy, cost, skinfeel and transparency on the skin.

In some embodiments, the total concentration of zinc oxide (exclusive of any surface treatment agents or dispersion/slurries/pastes excipients) in the formulation may be or may be equal to or not higher than 25%, e.g., equal to or not higher than 24%, 23%, 22%, 21%, 20%, 15%, or 10% by weight based on a total weight of the formulation. In some embodiments, the concentration of a first zinc oxide in the formulation may be equal to or not higher than about 25%, e.g., equal to or not higher than about 24%, about 23%, about 22%, about 21%, about 20%, about 19%, about 18%, about 17%, about 16%, about 15%, about 14%, about 13%, about 12%, about 11%, or about 10%, by weight, while the concentration of a second zinc oxide in the formulation may be equal to or not higher than about 15%, e.g., equal to or not higher than about 14%, about 13%, about 12%, about 11%, about 10%, about 9%, about 8%, about 7%, about 6%, about 5%, about 4%, about 3%, about 2%, about 1% or about 0%.

For example, in one variation, the formulation includes a first zinc oxide treated with triethoxycaprylylsilane. Such first zinc oxide treated with triethoxycaprylylsilane may have a first weight ratio of zinc oxide to triethoxycaprylylsilane of from 95:5 to 99:1. e.g., from 96:4 to 98:2 such as, e.g., 96.2:3.8. In other variations, the first zinc oxide treated with triethoxycaprylylsilane may separately or additionally have a median particle size (D50) of less than about 1800 nm, e.g., about 1790 nm. In yet other variations, the first zinc oxide exclusive triethoxycaprylylsilane may be present in an amount of about 25 wt % based on the total weight of the sunscreen formulation.

In other variations, the formulation may further include a second zinc oxide treated with triethoxycaprylylsilane. Such second zinc oxide treated with triethoxycaprylylsilane may have a second weight ratio of zinc oxide to triethoxycaprylylsilane of from about 95:5 to about 99:1, e.g., from about 96:4 to about 98:2, such as about 96.7:3, and/or may have a median particle size (D50) of about 2900 nm, e.g., about 2880 nm.

In yet other variations of the foregoing formulation, the first zinc oxide exclusive triethoxycaprylylsilane may be present in an amount of about 18 wt % and/or the second zinc oxide exclusive triethoxycaprylylsilane may be present in an amount of about 7 wt % based on the total weight of the sunscreen formulation.

### Film Formers

In certain embodiments, one or more film formers may be present in the compositions described herein. In some variations, a combination of two or more film formers may be used.

In some variations, the film formers may include one or both of bis-octyldodecyl dimer dilinoleate/propanediol copolymer and octyldodecyl/glyceryl hydroxy stearate dilinoleate dimethicone copolymer.

In one variation, the compositions described herein may further include a combination of bis-octyldodecyl dimer dilinoleate/propanediol copolymer and octyldodecyl/glyceryl hydroxy stearate dilinoleate dimethicone copolymer. The use of such combination of film formers was unexpectedly found to usually contribute to water resistance of up to at least 80 minutes. The use of such film formers usually contributed to the desirable aesthetic qualities of the final product, and also acted together to layer sunscreen films on the skin.

In some embodiments, the one or more film formers may be present in an amount of equal to or less than about 7.5%. e.g., equal to or less than about 5%, about 4%, about 3%, about 2%, about 1%, about 0.5%, about 0.25%, or about 0.1% by weight. In certain embodiments, the one or more film formers may be present in an amount of about 3 wt % based on the total weight of the sunscreen formulation.

In some variations, the one or more film formers may be present in a dry matter content ranging from 0.1% to 60%, e.g., from 0.5% to 40%, or from 1% to 30% by weight relative to the total weight of the composition.

In one embodiment, the one or more film may be a polymer capable of forming, on its own or in the presence of a film-forming aid, a continuous and adherent film on a support, for instance on keratinous materials.

In some variations, the one or more film formers used may be capable of forming a hydrophobic film. In certain variations, the one or more film formers may be polymers capable of forming a hydrophobic film having a solubility in water at 25° C. of less than 1% by weight.

In other variations, the one or more film formers may be synthetic polymers of the free-radical type or of the polycondensate type, polymers of natural origin and mixtures thereof. Free-radical film-forming polymers include polymers obtained by polymerization of monomers with for instance, ethylenic unsaturation, each monomer being capable of homopolymerizing (in contrast to polycondensates).

In other variations, the one or more film formers may be of the free-radical type, for example, vinyl polymers or copolymers. Examples include acrylic polymers. In one variation, the vinyl film-forming polymers may result from the polymerization of ethylenically unsaturated monomers having at least one acid group and/or esters of these acid monomers and/or amides of these acid monomers. In another variation, as a monomer carrying an acid group, there may be used C.B-ethylenic unsaturated carboxylic acids such as acrylic acid, methacrylic acid, crotonic acid, maleic acid and itaconic acid. For example, (meth)acrylic acid and itaconic acid may be used. In yet another variation, the esters of acid monomers may be chosen from the esters of (meth)acrylic acid (also called (meth)acrylates), for example alkyl, such as C-Co, for instance C—C, alkyl, (meth)acrylates, aryl, such as Co-Co aryl, (meth)acrylates, hydroxyalkyl, for instance C—C hydroxyalkyl, (meth)acrylates. Examples of alkyl (meth)acrylates may include methyl methacrylate, ethyl methacrylate, butyl methacrylate, isobutyl methacrylate, 2-ethyl hexyl methacrylate, lauryl methacrylate and cyclohexyl methacrylate. Examples of hydroxyalkyl (meth)acrylates may include hydroxyethyl acrylate, 2-hydroxypropyl acrylate, hydroxyethyl methacrylate and 2-hydroxypropyl methacrylate. Examples of aryl (meth)acrylates may include benzyl acrylate and phenyl acrylate. In certain variations the alkyl group of the esters may be either fluorinated or perfluorinated (e.g., some or all of the hydrogen atoms of the alkyl group may be substituted with fluorine atoms). Examples of amides of the acid monomers may include (meth)acrylamides, such as N-alkyl(meth)acrylamides, and for instance, of a C—C alkyl. Examples of N-alkyl(meth)acrylamides may include N-ethylacrylamide, N-t-butylacrylamide. N-t-octylacrylamide and N-undecylacrylamide.

In some variations, the vinyl film formers may also result from the homopolymerization or copolymerization of monomers chosen from vinyl esters and styrene monomers. For example, these monomers may be polymerized with acid monomers and/or their esters and/or their amides, such as those mentioned above. Examples of vinyl esters may include vinyl acetate, vinyl neodecanoate, vinyl pivalate, vinyl benzoate and vinyl t-butylbenzoate. Examples of styrene monomers may include styrene and alpha-methylstyrene. In other variations, any acrylic and vinyl monomers (including the monomers modified by a silicone chain) may be used.

In other variations, the film formers may be film-forming polycondensates, such as polyurethanes, polyesters, polyester amides, polyamides, epoxy ester resins, and polyureas. In one variation, the polyurethanes may be chosen from anionic, cationic, nonionic and/or amphoteric polyurethanes, polyurethane-acrylics, polyurethane-polyvinylpyrrolidones, polyester-polyurethanes, polyether-polyurethanes, polyureas, and polyurea-polyurethanes. In certain variations, the polyesters may be obtained by polycondensation of dicarboxylic acids with polyols, such as diols. In yet other variations, the dicarboxylic acids may be aliphatic, alicyclic and/or aromatic. Suitable acids may include, for example, oxalic acid, malonic acid, dimethylmalonic acid, succinic acid, glutaric acid, adipic acid, pimelic acid, 2.2-dimethylglutaric acid, azelaic acid, suberic acid, sebacic acid, fumaric acid, maleic acid, itaconic acid, phthalic acid, dodecanedioic acid, 1.3-cyclohexanedicarboxylic acid, 1,4-cyclohexanedicarboxylic acid, isophthalic acid, terephthalic acid, 2.5-norbornanedicarboxylic acid, diglycolic acid, thiodipropionic acid, 2.5-naphthalenedicarboxylic acid and 2.6-naphthalenedicarboxylic acid. These dicarboxylic acid monomers described herein may be used alone or in combination with at least two dicarboxylic acid monomers. Examples of monomers that may be used are phthalic acid, isophthalic acid and terephthalic acid. In other variations, the diols may be chosen from aliphatic, alicyclic and/or aromatic diols. For example, the diols may be chosen from: ethylene glycol, diethylene glycol, triethylene glycol, 1,3-propanediol, cyclohexanedimethanol and 4-butanediol. Examples of polyols may include glycerol, pentaerythritol, sorbitol, and trimethylolpropane.

In some variations, the polyester amides may be obtained in a manner similar to the polyesters, by polycondensation of diacids with diamines or amino alcohols. As diamines, there may be used ethylenediamine, hexamethylenediamine, meta- and/or para-phenylenediamine. As aminoalcohols, monoethanolamine may be used. In certain variations, the polyester may, in addition, comprise at least one monomer carrying at least one —SOM group, wherein M is chosen from hydrogen atoms, ammonium ions and metal ions (such as for example Na, Li, K, Mg, Ca, Cu, Fe ions). In other variations, the at least one monomer may be a bifunctional aromatic monomer comprising such an —SOM group. In certain variations, the aromatic ring of the bifunctional aromatic monomer carrying an —SOM group as described above may be chosen, for example, from benzene, naphthalene, anthracene, diphenyl, oxydiphenyl, sulphonyl diphenyl and methylenediphenyl rings, sulphoisophthalic acid, sulphoterephthalic acid, sulphophthalic acid, and 4-sulphonaphthalene-2,7-dicarboxylic acid.

In other variations, copolymers may be used. In certain variations, the copolymers are based on isophthalate or sulphoisophthalate. In certain variations, the copolymers may be obtained by condensation of diethylene glycol, cyclohexanedimethanol, isophthalic acid and sulphoisophthalic acid. In certain variations, the optionally modified polymers of natural origin may be chosen from shellac resin, sandarac gum, dammars, elemis, copals, cellulosic polymers and mixtures thereof.

In some embodiments, the film formers may be present in the form of particles in aqueous dispersion, generally known as latex or pseudolatex. The techniques for preparing these dispersions are well known to persons skilled in the art.

In other variations, the film formers may be present as an aqueous dispersion of film-forming polymers. In certain variations, the film formers may be dispersions of polymers resulting from the free-radical polymerization of one or more free radical monomers inside and/or partly at the surface, of preexisting particles of at least one polymer chosen from polyurethanes, polyureas, polyesters, polyesteramides and/or alkyls. These polymers are generally called hybrid polymers.

In other variations, the one or more film formers may be water-soluble polymers and may be present in the aqueous phase of the composition in solubilized form. Examples of film-forming water-soluble polymers may include proteins. In one variation, the proteins may be of plant origin (such as wheat or soya bean proteins), proteins of animal origin (such as keratins, for example keratin hydrolysates and sulphonic keratins), as well as anionic, cationic, amphoteric or nonionic polymers of chitin or chitosan. In other variations, the polymers may be cellulose polymers such as hydroxyethyl cellulose, hydroxypropyl cellulose, methyl cellulose, ethyl hydroxyethyl cellulose, carboxymethyl cellulose, and quaternized derivatives of cellulose. In yet other variations, the polymers are acrylic polymers or copolymers such as poly acrylates or polymethacrylates. In yet other variations, the polymers may be vinyl polymers, such as polyvinylpyrrolidones, copolymers of methyl vinyl ether and maleic anhydride, copolymers of vinyl acetate and crotonic acid, copolymers of vinylpyrrolidone and vinyl acetate; copolymers of vinylpyrrolidone and caprolactam; polyvinyl alcohol: In other variations, the polymers may be optionally modified polymers of natural origin, such as gum arabic, guar gum, xanthan derivatives, karaya gum, glycoaminoglycans, hyaluronic acid and its derivatives; shellac resin, sandarac gum, dammars, elemis, copals; alginates and carrageenans; deoxyribonucleic acid; and muccopolysaccharides such as chondroitin sulphates.

In one embodiment, the one or more film formers may be selected from one or more of vinyl polymers, vinyl copolymers (such as acrylic polymers or acrylates), fluorinated or perfluorinated acrylates, acrylamides, vinyl esters, styrene esters, silicone-modified vinyl polymers, vinyl copolymers, acrylates, acrylamides, other vinyl esters, styrene esters, polyurethanes, polyesters, polyester amides, polyamides, epoxy ester resins, polyureas, optionally modified polymers of natural origin, shellac resin, sandarac gum, dammars, elemis, copals, cellulosic polymers, hydrolyzed starches, latexes, abietates, hybrid polymers, proteins, anionic, cationic, amphoteric or nonionic polymers of chitin or chitosan, cellulose polymers, and pullulan.

In other variations, the one or more film formers may be selected from one or more of anionic, cationic, nonionic and/or amphoteric polyurethanes, polyurethane-acrylics, polyurethane-polyvinylpyrrolidones, polyester-polyurethanes, polyether-polyurethanes, polyureas, and polyurea-polyurethanes. In one variation, the one or more film formers may be diols.

In another variation, the one or more film formers may be selected from one or more of wheat or Soya bean proteins; proteins of animal origin such as keratins, for example keratin hydrolysates and sulphonic keratins.

In yet another variation, the one or more film formers may be selected from one or more of hydroxyethyl cellulose, hydroxypropyl cellulose, methyl cellulose, ethyl hydroxyethyl cellulose, carboxymethyl cellulose, and quaternized derivatives of cellulose.

### SPF Boosters

In certain embodiments, one or more SPF boosters are present in the compositions described herein. In some variations, a combination of SPF boosters is used.

Non-limiting examples of SPF boosters include butyloctyl salicylate, ethylhexyl methoxycrylene, styrene/acrylates copolymer (and) acrylates copolymer, glycogen, acrylates/methacryloyloxyethyl phosphate copolymer, dimethyl capramide, neopentyl glycol diethylhexanoate (and) neopentyl glycol diisostearate, Daucus carota sative (carrot) root extract (and) Helianthus annuus (sunflower) seed oil, argania spinose kernel oil (and) tocopheryl acetate (and) bisabolol, PVP (and) VP/Eicosene copolymer, VP/eicosene copolymer, hydrophobically modified starches, silica beads, PMMA beads, borosilicate beads, polyurethane beads, diatomaceous earth, bentonite and hectorite clays and any combination thereof.

In some embodiments, the one or more SPF boosters may be present in an amount of equal to or less than about 10%, e.g., equal to or less than about 9%, about 8%, about 7%, about 6%, about 5%, about 4%, about 3%, about 2%, about 1%, about 0.5%, about 0.2%, or about 0.1% by weight. In certain embodiments, the one or more SPF boosters may be present in an amount of about 6 wt % based on the total weight of the sunscreen formulation. In certain embodiments, the one or more SPF boosters may be present in an amount ranging from 0.1 wt % to 8 wt %.

### Emulsification and Stabilization Components

In certain embodiments, one or more emulsifiers may be present in the compositions described herein. In other embodiments, one or more humectants and other stabilizers may be present in the compositions described herein. In some variations, a combination of emulsifiers, humectants and other stabilizers may be used.

In some variations, the compositions may further comprise a silicone-based emulsifier. Non-limiting examples of silicone-based emulsifiers include cetyl PEG/PPG 10/1 Dimethicone, cetyl diglyceryl tris(trimethylsiloxy) silylethyl dimethicone, bis-PEG/PPG-14/14 dimethicone (and) dimethicone, dimethicone (and) dimethicone/vinyl dimethicone crosspolymer (and) PEG-10 dimethicone, cyclopentasiloxane (and) dimethicone (and) dimethicone/vinyl dimethicone crosspolymer (and) PEG-10 dimethicone, lauryl PEG-8 dimethicone. PEG-10 dimethicone, dimethicone (and) caprylyl dimethicone ethoxy glucoside, cyclopentasiloxane (and) caprylyl dimethicone ethoxy glucoside, dimethicone (and) PEG/PPG-18/18 dimethicone, cyclopentasiloxane (and) PEG/PPG-20/15 dimethicone, cyclopentasiloxane (and) PEG-10 dimethicone (and) Bentonite (and) distearyldimonium chloride, dimethicone (and) PEG/PPG-18/18 dimethicone, PEG-8 dimethicone, PEG/PPG-19/19 dimethicone (and) C13-16 isoparaffin (and) C10-13 isoparaffin, mineral oil (and) PEG-15/Lauryl dimethicone crosspolymer, bis-isobutyl PEG/PPG-10/7/dimethicone copolymer, sorbitan olivate, cyclopentasiloxane (and) PEG/PPG-18/18 dimethicone and any combination thereof. Non-silicone emulsifiers such as, e.g., sorbitan olivate, steareth-20, steareth-2, steareth-21, methyl glucose dioleate polyglyceryl-4 isostearate (and) coco-caprylate/caprate (and) hectorite, polyglyceryl-2 dipolyhydroxystearate may also be used in the compositions of the present invention.

Suitable humectants may include moisturizing humectants (such as glycerin, hydroxyethyl urea, betaine, sodium PCA, sodium-L-Lactate and propanediol), antimicrobial potentiating humectants (such as 1,2 pentylene glycol, and 1,2 hexanediol), humectant solvents (such as 1,2 hexanediol, PEG-4, 8, dipropylene glycol, 1,2 pentylene glycol, propanediol, 1,3 butylene glycol, 2-methyl-1,3-propanediol and propylene glycol) and natural based humectants (such as glycerin, pentylene glycol, 1,3 butylene glycol, propanediol, sodium PCA, sodium-L-Lactate and betaine).

Examples of suitable stabilizers for the instant compositions include chlorides (such as sodium chloride, potassium chloride, and magnesium chloride), carbonates (such as sodium carbonate, potassium carbonate, magnesium carbonate and propylene carbonate), sulfates (such as sodium sulfate, zinc sulfate, and magnesium sulfate), silica, polymeric thickeners, natural gums, olefin resins, waxes, and any combination thereof.

In some variations, a photostabilizer is used. In certain embodiments, the photostabilizers may also boost the SPF.

### Skinfeel Enhancers

In certain embodiments, one or more skinfeel enhancers are present in the compositions described herein. In some variations, a combination of the skinfeel enhancers described herein is used.

Suitable skinfeel enhancers may include elastomers and silicones. In some variations, the skinfeel enhancers are silicon elastomer blends, or polydimethylsiloxane fluids. In certain variations, the skinfeel enhancers may be silicone crosspolymers, dimethicone crosspolymers, dimethicone/vinyl dimethicone crosspolymers, polysilicone-modified silicones, hydrocarbon/silicone crosspolymers, hydrocarbon/dimethicone crosspolymers, hydrocarbon crosspolymers, alkyl/silicone crosspolymers, alkyl/dimethicone crosspolymers, or alkyl crosspolymers. In other variations, the skinfeel enhancers may be dimethicones, cyclic siloxanes, linear silicones, organofunctional silicones, or organofunctional polydimethylsiloxanes. In yet other variations, the skinfeel enhancers may be squalane or hemisqualane.

In some variations, the skinfeel enhancers used in the compositions described herein helps to mitigate product drag, impart dry cushion during and after rubout, as well as help to anchor the sunscreen film on the skin, and improve product spreadability and drytime.

### Excipients

In certain embodiments, the composition may further comprise one or more excipients. Suitable excipients may include compounds that can help to improve skinfeel, viscosity, stability, SPF boosting capability, and cost. Suitable excipients may include, for example, esters (such as ethylhexyl isononanoate, C12-15 Alkyl Benzoate, and isopropyl palmitate), beeswax.

### Vitamins

In certain embodiments, the compositions may further comprise one or more vitamins. For example, in some variations, vitamin E may be present in the compositions described herein. In some embodiments, vitamin E may be present at about 2%, about 1%, about 0.5%, about 0.25%, about 0.1%, about 0.5%, about 0.25%, or about 0.01% by weight. In one variation, the vitamin E may be present at about 0.25% by weight.

### Preservation System Components

In certain embodiments, the compositions may further comprise one or more preservation system components. Such preservation system components may include ingredients that provide additional protection against bacterium growth, such as biostatic agents.

### Other Ingredients

In certain embodiments, the compositions may further comprise botanicals, such as plant extracts.

In some variations, the compositions may be formulated as sunscreen lotions or creams. In other variations, the compositions may be formulated as sunscreen sprays.

### Composition Properties

The compositions described herein may have one or more of the following properties.

In one variation, the sunscreen formulations described herein have a Sun Protection Factor (SPF) of at least about 50. In some embodiments, the sunscreen formulations may have a SPF of at least about 55 or at least about 60. In some embodiments, the sunscreen formulations may have a SPF in a range from about 50 to about 70, or from about 55 to about 60.

In another variation, the sunscreen formulations described herein may have a UVA Protection Factor (UVAPF) of at least about 16, at least about 17, at least about 18, at least about 19, at least about 20, at least about 21, or at least about 22. In some embodiments, the sunscreen formulations may have a UVAPF in a range from about 16 to about 20, or about 16 to about 17.

In yet another variation, the sunscreen formulations described herein may have a water resistance of up to 80 minutes, for example, at least about 40 minutes, at least about 50 minutes, at least about 60 minutes or at least about 70 minutes. In some embodiments, the sunscreen formulations may have a water resistance in a range from about 40 to about 80 minutes, or from about 40 to about 60 minutes.

In another variation, the sunscreen formulations described herein may be tear-free.

In some variations, the compositions may have two or more, three or more, four or more, or all of the properties listed above.

### Manufacturing Methods

Provided herein are also methods to produce the compositions described herein. In some aspects, provided is a method of manufacturing the sunscreen formulations herein, comprising: mixing one or more film formers and one or more SPF boosters to provide a silicone/oil mixture; adding to the first mixture no more than 25 wt % zinc oxide based on a total weight of the final inverted emulsion, wherein the zinc oxide is preferably surface treated; and combining the silicone/oil mixture with an aqueous mixture to provide the hybrid water-in-silicone/oil inverted emulsion.

In some embodiments, the method may further comprise continuously stirring the silicone/oil mixture when the aqueous mixture is added. In some embodiments, the method may further comprise homogenizing and milling during stirring when the silicone/oil mixture and the aqueous mixture are mixed, and/or when the silicone/oil mixture is provided. Such continuous stirring and additional steps may help to disperse the zinc oxide and improve incorporation of the actives into the emulsion.

### EXAMPLES

The following Examples are merely illustrative and are not meant to limit any aspects of the present disclosure in any way.

### Example 1: Development of Sunscreen Formulations

This example demonstrates the preparation and characterization of four exemplary sunscreen formulations, made up of surface-treated zinc oxide in a hybrid water-in-silicone/oil inverted emulsion system.

Preparation of Sunscreen Formulation. Four exemplary formulations were prepared by combining ingredients of Part A and Part B in the concentrations and quantities listed in Table 1 below. Pre-weigh zinc oxide into an appropriate container. Pre-weigh Phase B ingredients in a suitable beaker and impeller-stir to uniformity at room temperature. Combine the remaining Part A ingredients in a separate appropriate size beaker which will be used as the main vessel. Stir vessel contents slowly with an impeller while heating the Phase A minus zinc oxide powders to 70-85° C. When vessel contents reach 70° C., slowly introduce zinc oxide powders into vessel. Maintaining appropriate temperature and vessel coverage (to prevent evaporation), increase speed or apply homogenization to ensure full incorporation of the powders and achieve a uniform composition. Alternatively, the zinc oxide powders can be introduced into the vessel under homogenization. When Part A is uniform, slowly add precombined Part B under adequate agitation. Adjust mixing speed or homogenization to allow full incorporation of Part B into the batch. When fully incorporated, adjust mixing speed down, keep batch covered, remove from heat, and cool to 38° C. or less.

TABLE 1
Summary of ingredients

| | | I | II | III | IV |
|---|---|---|---|---|---|
| | | (wet | (wet | (wet | (wet |
| | Ingredient | wt %) | wt%) | wt%) | wt%) |
| Part A | Zinc oxide (and) | 25.00 | 18.00 | 25.00 | 18.00 |
| | Triethoxycaprytylsilane | | | | |
| | 96.2:3.8 | | | | |
| | D50 = 1790 mm | | | | |
| | Zinc oxide (and) | 7.00 | | 7.00 | |
| | Triethoxycaprylylsilane | | | | |
| | 96.67:3.33 | | | | |
| | D50 = 2880 nm | | | | |
| | Bis-octyldodecyl Dimer | 2.00 | 2.00 | 2.00 | 2.00 |
| | Dilinoleate/Propanediol | | | | |
| | Copolymer | | | | |
| | Octyldodecyl/Glyceryl | 1.00 | 1.00 | 1.00 | 1.00 |
| | Hydroxy Stearate | | | | |
| | Dilinoleate Dimethicone | | | | |
| | Copolymer | | | | |
| | Butyloctyl Salicylate | 5.0 | 5.00 | 5.00 | 5.00 |
| | Ethythexyl | 1.00 | 1.00 | 1.00 | 1.00 |
| | Methoxycrylene | | | | |
| | Cetyl PEG/PPG 10/1 | 2.50 | 5.00 | 2.50 | 3.00 |
| | Dimethicone | | | | |
| | Hydroxyacetophenone | 0.50 | 0.50 | 0.50 | 0.50 |
| | Ethythexyl | 5.00 | 5.00 | 5.00 | 5.00 |
| | Isononanoate | | | | |
| | C12-15 Alkyl Benzoate | 5.00 | 8.00 | 5.00 | 8.00 |
| | Isopropyl Palmitate | 5.00 | 8.00 | 5.00 | 8.00 |
| | Beeswax | 0.50 | 0.50 | 0.50 | 0.50 |
| | Cyclopentasiloxane and | 3.00 | 3.00 | 3.00 | 3.00 |
| | PEG-12 Dimethicone | | | | |
| | Crosspolymer | | | | |
| | Dimethicone, NF 350 | 5.00 | | 5.00 | |
| | cst | | | | |
| | Dimethyl Siloxane, | | 2.00 | | 2.00 |
| | Trimethylsiloxy- | | | | |
| | Terminated | | | | |
| | Vitamin E, USP | | | 0.25 | 0.25 |
| Part B | Purified Water, USP | 35.60 | 32.10 | 34.35 | 30.85 |
| | Propylene Glycol, USP | 3.00 | 3.00 | 3.00 | 3.00 |
| | Sodium Chloride, USP | 0.40 | 0.40 | 0.40 | 0.40 |
| | 1,2-Hexanediol (and) | 0.50 | 0.50 | 0.50 | 0.50 |
| | Caprylyl Glycol | | | | |
| | Plant extract | | | 1.00 | 1.00 |

Both formulations I and II passed SPF 50 VWR and UVAPF testing. Formulation I showed an average SPF rating of 52.23 and a UVAPF rating of 20.70, while formulation II showed an average SPF rating of 51.52 and a UVAPF rating of 22.72. It was also observed that formulation I was more stable and less whitening than formulation II.

Formulations III and IV, both containing antioxidant and botanicals packages were also tested. Both formulations passed SPF testings (SPF>50 and UVAPF>20). Formulation III whitened less, and provided better skinfeel than formulation IV.

1.-63. (canceled)

64. A sunscreen formulation having a SPF rating of at least 50, wherein the formulation comprises:
not more than 25 wt % of one or more zinc oxides, based on a total weight of the sunscreen formulation, the one or more zinc oxides being surface-treated;
one or more film formers; and
one or more SPF boosters,
and wherein the sunscreen formulation does not comprise any organic sunscreen active ingredients.

65. The sunscreen formulation of claim 64, wherein the one or more zinc oxides comprises at least one zinc oxide surface-treated with an ethoxylated alkyl silane.

66. The sunscreen formulation of claim 65, wherein the surface-treated zinc oxide comprises at least one zinc oxide surface-treated with triethoxycaprylylsilane.

67. The sunscreen formulation of claim 64, wherein the one or more surface-treated zinc oxides comprise a first zinc oxide surface-treated with an ethoxylated alkylsilane.

68. The sunscreen formulation of claim 67, wherein the first zinc oxide is surface-treated with triethoxycaprylylsilane and has a first weight ratio of zinc oxide to triethoxycaprylylsilane of about 96.2:3.8.

69. The sunscreen formulation of claim 67, wherein the first zinc oxide is surface-treated with triethoxycaprylylsilane and has a first median particle size (D50) of about 1790 nm.

70. The sunscreen formulation of claim 67, wherein the first zinc oxide is present in an amount of about 25 wt % based on the total weight of the sunscreen formulation.

71. The sunscreen formulation of claim 67, wherein the one or more surface-treated zinc oxides further comprise a second zinc oxide surface-treated with an ethoxylated alkylsilane.

72. The sunscreen formulation of claim 71, wherein the second zinc oxide is surface-treated with triethoxycaprylylsilane and has a second weight ratio of zinc oxide to triethoxycaprylylsilane of about 96.6:3.3.

73. The sunscreen formulation of claim 71, wherein the second zinc oxide surface-treated with triethoxycaprylylsilane has a second median particle size (D50) of about 2880 nm.

74. The sunscreen formulation of claim 71, wherein the first zinc oxide is present in an amount of about 18 wt % and the second zinc oxide is present in an amount of about 7 wt % based on the total weight of the sunscreen formulation.

75. The sunscreen formulation of claim 64, wherein the one or more film formers comprise bis-octyldodecyl dimer dilinoleate/propanediol copolymer.

76. The sunscreen formulation of claim 64, wherein the one or more film formers comprise octyldodecyl/glyceryl hydroxy stearate dilinoleate dimethicone copolymer.

77. The sunscreen formulation of claim 64, wherein the one or more film formers comprise bis-octyldodecyl dimer dilinoleate/propanediol copolymer and octyldodecyl/glyceryl hydroxy stearate dilinoleate dimethicone copolymer.

78. The sunscreen formulation of claim 64, wherein the one or more film formers are present in an amount of from about 0.1 to 60 wt % based on the total weight of the sunscreen formulation.

79. The sunscreen formulation of claim 64, wherein the one or more SPF boosters comprise butyloctyl salicylate.

80. The sunscreen formulation of claim 64, wherein the one or more SPF boosters comprise ethylhexyl methoxycrylene.

81. The sunscreen formulation of claim 64, wherein the one or more SPF boosters comprise butyloctyl salicylate and ethylhexyl methoxycrylene.

82. A sunscreen formulation, wherein the formulation comprises:
at least one zinc oxide surface-treated with triethoxycaprylylsilane;
bis-octyldodecyl dimer dilinoleate/propanediol copolymer;
octyldodecyl/glyceryl hydroxy stearate dilinoleate dimethicone copolymer; and
at least one SPF booster
and wherein the sunscreen formulation is a water-in-silicone/oil inverted emulsion, and contains not more than 25 wt % zinc oxide based on the total weight of the sunscreen formulation.

83. A water-in-silicone/oil inverted emulsion, wherein the formulation comprises:
not more than 25 wt % zinc oxide based on a total weight of the emulsion, wherein the zinc oxide is surface treated;
one or more film formers; and
one or more SPF boosters,
and wherein the inverted emulsion does not comprise any organic sunscreen active ingredients.
